(19) United States
(12) Patent Application Publication
(10) Pub. No.: US 2023/0093682 A1
(43) Pub. Date: Mar. 23, 2023

(54) METHODS FOR MANUFACTURING SILICON NITRIDE MATERIALS

(71) Applicant: SINTX TECHNOLOGIES, INC., Salt Lake City, UT (US)

(72) Inventors: Bryan J. McEntire, Salt Lake City, UT (US); Ryan M. Bock, Salt Lake City, UT (US); Clayton Ashcroft, Salt Lake City, UT (US); Bhajanjit Singh Bal, Salt Lake City, UT (US)

(21) Appl. No.: 17/934,402

(22) Filed: Sep. 22, 2022

Related U.S. Application Data
(60) Provisional application No. 63/247,091, filed on Sep. 22, 2021.

(51) Int. Cl. C04B 35/584; A61L 27/10

(52) U.S. Cl. CPC C04B 35/584; A61L 27/10; C04B 2235/5252; C04B 2235/665; A61L 2400/18; A61L 2420/02

(57) ABSTRACT

The present disclosure relates to the manufacture of silicon nitride implants with increased surface roughness and porosity.

### CROSS-REFERENCE TO RELATED APPLICATIONS

This application claims priority to U.S. Patent Application No. 63/247,091, filed on Sep. 22, 2021, the contents of which are incorporated herein by reference in their entirety.

### FIELD

The present disclosure relates to the manufacture of silicon nitride osteogenic implants with increased surface roughness and porosity. Therefore, the present disclosure relates to the fields of medicine, materials science, and machining.

### BACKGROUND

It is becoming increasingly clear that the osteogenic ability of any biomaterial is governed by several critical surface properties including its chemical composition, wettability, electrical charge, crystallinity, elution behavior, and topography. However, much of the historical literature on the biological properties of biomaterials has either ignored these characteristics or have focused solely on one aspect at the exclusion of the others. In the beginning, the primary concerns for abiotic materials rested solely on their biocompatibility and mechanical properties. Historically, titanium (cp-Ti and Ti6AI4V-ELI) and polyetheretherketone (PEEK) spinal implants were utilized without significant characterization or even a basic understanding of their surface functional properties; and while silicon nitride (Si₃N₄) is a comparatively recent addition to the library of spinal materials, it too was cleared for implantation solely based on the validation of its biocompatibility and mechanical properties. However, development of an effective arthrodesis device requires concurrent optimization of all the important surface properties.

Titanium alloys have been around since just after World War II and actively used as implants since the 1970s. Biomedical titanium is essentially bioinert because of a thin passivation layer of titanium dioxide (TiO₂) which prevents significant biochemical interactions. However, when titanium is placed in vivo, the normal oxide layer (˜2 to 7 nm) thickens and incorporates bio-minerals (i.e., Na, Ca, etc.). Depending on the local environment, rutile and/or anatase form along with various non-stoichiometric titanium oxides and hydroxyls. Nevertheless, the growth of this layer is diffusion-limited, and it eventually becomes a stable corrosion barrier to bodily fluids.

Biomedical PEEK was introduced in the 1990s and rapidly gained acceptance as a spinal spacer because of its lower cost, favorable modulus, and ease of use. Its rise in popularity was accelerated because of subsidence concerns associated with stiffer materials. It was hypothesized that spacer materials with increased modulus might lead to stress shielding of adjacent bone thereby discouraging fusion. In fact, the Young's modulus of PEEK more closely approximates that of cortical bone than titanium (PEEK E=4 GPa; Ti E=105 GPa, Bone E=7-26 GPa). It was reasoned that matching the modulus of the implant to bone might decrease the risk of spacer subsidence. However, other studies have shown that the initial and long-term mechanical stability of a spinal spacer may be more dependent upon its overall geometry than its elastic modulus. Additionally, PEEK does not integrate into adjacent host bone, and it is not visible on plain x-rays.

Silicon nitride has proven to be an effective arthrodesis device. The surface chemistry (i.e., elution of ammonia and silicic acid) is likely an important factor in its osseointegrative and bacteriostatic effectiveness. However, results from in vitro and small animal studies have yet to be confirmed in large animal models and human clinical trials. It is suspected that this is due to a sub-optimal macro- and micro-surface morphology and an inadequate presence of bone-promoting minerals.

### SUMMARY OF THE DISCLOSURE

Disclosed herein are methods for manufacturing silicon nitride implants having enhanced osseointegrative effectiveness. The disclosed method includes providing a silicon nitride green body, increasing the surface roughness of the silicon nitride green body, increasing the porosity of the silicon nitride green body, and then sintering the silicon nitride green body to obtain a silicon nitride implant.

The step of increasing the surface roughness of the silicon nitride green body may be performed by laser etching. The Sₐ of the silicon nitride implant may be less than about 100 μm. In some examples, the Sₐ of the silicon nitride implant may be about 60 μm to about 90 μm.

The step of increasing the porosity of the silicon nitride green body may be performed by peck drilling and/or laser etching. The pores of the silicon nitride green body may each have a diameter of about 400 μm to about 600 μm.

The method may further include adding an osteogenic coating after the sintering step. The osteogenic coating may be selected from the group consisting of SiYAION, NanoHA®, 45S5 Bioglass®, hydroxyapatite, and combinations thereof.

### BRIEF DESCRIPTION OF THE DRAWINGS

The present patent or application file contains at least one drawing executed in color. Copies of this patent or patent application publication with color drawing(s) will be provided by the Office upon request and payment of the necessary fee.

FIGS. 1A-1B show an example bit map used to map an increase in surface roughness.

FIGS. 2A-2G show an example bit map used to map an increase in surface roughness.

FIGS. 3A-3H show fluorescence microscopy evaluation of osteocalcin production by osteoblastic activity after 7-days of incubation. FIG. 3A shows the osteocalcin production on as-fired silicon. FIG. 3B shows the osteocalcin production on N₂-annealed Si₃N₄. FIG. 3C shows the osteocalcin production on 0.1 vol % SiYAION glazed Si₃N₄. FIG. 3D shows the osteocalcin production on NanoHA® coated Si₃N₄. FIG. 3E shows the osteocalcin production on machined Ti6AI4V-ELI. FIG. 3F shows the osteocalcin production on 45S5 Bioglass®. FIG. 3G shows osteocalcin production on Machined PEEK. FIG. 3H shows the osteocalcin production on 10 vol. % SiYAION glazed Si₃N₄.

FIGS. 4A-4B show the hydroxyapatite volume deposited by action of SaOS-2 osteoblast cells per surface unit of several different Si₃N₄-treated surfaces. The results were independently evaluated by two operators: Operator 1 (FIG. 4A) and Operator 2 (FIG. 4B).

FIGS. 5A-5B show the surface topographies of Si₃N₄ on an as-fired surface (FIG. 5A) and a machined surface (FIG. 5B).

FIGS. 6A-6C show examples of silicon nitride implants with increased surface roughness and porosity.

FIG. 7 shows a collage of scanning electron micrographs detailing the macro-, micro-, meso-, and nano-structure of a laser textured silicon nitride implant.

FIGS. 8A-8B show white-light interferometry surface roughness measurements of as-fired Si₃N₄ (FIG. 8A) and laser etched and as-fired Si₃N₄ (FIG. 8B).

FIGS. 9A-9C show an implant of the present disclosure. FIG. 9A shows a perspective view of an implant of the present disclosure. FIG. 9B shows a top-down view of an implant of the present disclosure. FIG. 9C shows a side-view of an implant of the present disclosure.

FIGS. 10A-10B show the surface roughness profile of an implant of the present disclosure using a Trumpf laser. FIG. 10A shows a heat map depicting the relative height of an area of the implant. FIG. 10B shows the height profile along a linear path of the area shown in FIG. 10A.

FIGS. 11A-11B show the surface roughness profile of an implant of the present disclosure using a Forba machine. FIG. 11A shows a heat map depicting the relative height of an area of the implant. FIG. 11B shows the height profile along a linear path of the area shown in FIG. 11A.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, the term “silicon nitride” includes Si₃N₄, alpha- or beta-phase Si₃N₄, SiYAION, SiYON, SiAION, or combinations of these phases or materials.

As used herein, the term “surface roughness” has its general meaning ordinarily used in the art. Unless stated otherwise, surface roughness is measured in this disclosure by the surface roughness parameters “Rₐ” or “Sₐ”, which refer to the arithmetical mean deviation of the assessed 2D or 3D profile, respectively, and are measured in μm.

As used herein, the term “implant” refers to any biomedical implant suitable for being implanted in the body. Non-limiting examples of implants include intervertebral spacers or other spinal implants, orthopedic screws, plates, or other fixation devices, articulation implants in the spine, hip, knee, shoulder, ankle or phalanges, implants for facial or other reconstructive plastic surgery, dental implants, and the like.

Disclosed herein are methods of manufacturing silicon nitride implants with improved antimicrobial and osseointegrative capabilities. The method includes providing a silicon nitride green body, increasing the surface roughness and porosity of the silicon nitride green body, and then sintering the silicon nitride green body. The surface roughness may be increased at the macro and micro scale. By manipulating the topography of the silicon nitride green body (i.e., prior to densification), the micro- and nano-structure of the implant, which is only formed during densification, is preserved. It was surprisingly found that performing the macro roughening operation via peck drilling and/or by a laser in the green state preserves the micro and nano roughness that develops during sintering. A combination of both macro, micro, and nano roughness improves osseous integration of the implants.

### Surface Roughness

The method disclosed herein includes increasing the surface roughness of a silicon nitride green body. The surface morphology of an osteogenic implant, including the surface roughness, plays a vital role in the mechanism for osteointegration. By modifying the surface roughness of the green body, the micro- and nano-structured surface morphology that is generated during densification and hot isostatic pressing is preserved. Not only does the surface morphology relate to the biological mechanisms for osseointegration bony apposition, but surface roughness is also useful to surgeons placing the implants. The increased surface roughness allows surgeons to fixate implants more easily during surgery.

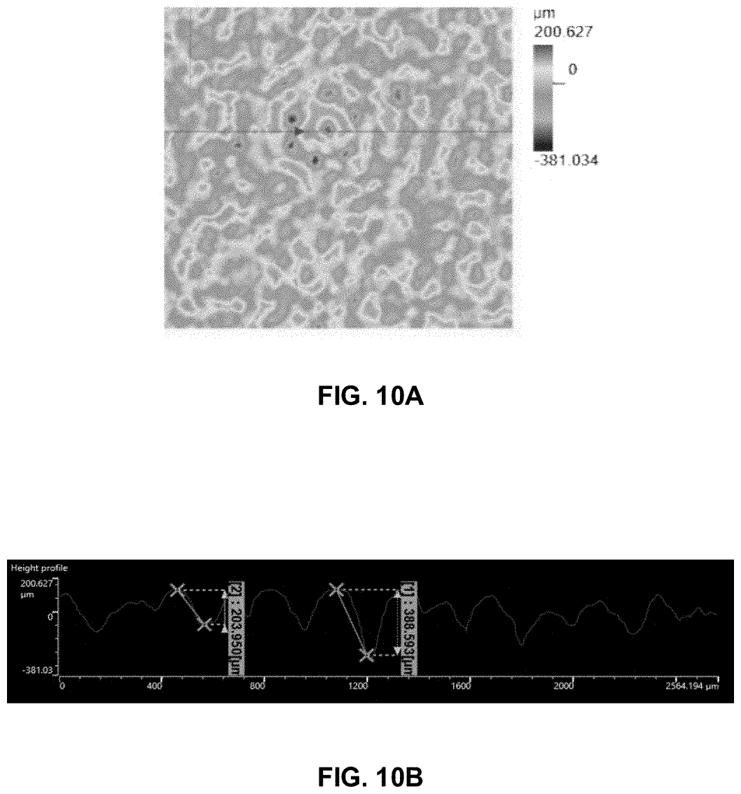

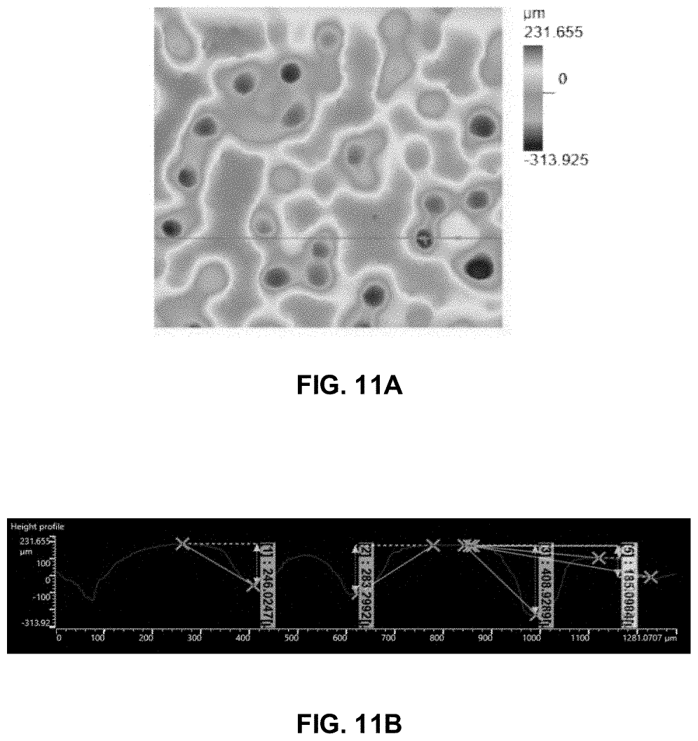

In some additional embodiments, an implant formed by the method disclosed herein may have a surface roughness measured by a Sₐ value of about 1 μm to about 100 μm. In some aspects, the implant may have a surface roughness measured by a Sₐ value of about 1 μm to about 10 μm, about 10 μm to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, or about 90 μm to about 100 μm. In some additional aspects, the implant may have a surface roughness measured by a Sₐ value of between about 20 μm to about 100 μm, or about 50 μm to about 90 μm. In still additional embodiments, the implant may have a surface roughness measured by a Sₐ value of about 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, or about 100 μm. In one example, the implant has a surface roughness measured by a Sₐ value of 90.6 μm, as seen in FIGS. 11A-11B. In another example, the implant has a surface roughness measured by a Sₐ value of 58.7 μm, as seen in FIGS. 10A-10B.

In some embodiments, the increase in surface roughness may be accomplished by laser etching the implant while it is in the green state. The average power of the laser may be between about 10 W to about 50 W. The frequency of laser pulses may be between about 1 kHz to about 250 kHz. The scan speed of the laser may be between about 50 mm per second to about 500 mm per second. The laser may have a line spacing of between about 20 μm to about 500 μm. The laser etching may be completed after about two to about six repetitions. The laser may be capable of achieving an engraving depth of about 50 μm to about 600 μm. In some embodiments, laser etching increases surface roughness by etching a pattern. Non-limiting examples of patterns include dimpled, cross hatches, parallel grooves, wave cross hatches, and geometric cross hatches. In some embodiments, the laser etching increases surface roughness by etching a pattern based on a predefined bit map. In some aspects, the bit map may consist of a plurality of dots organized randomly in the bitmap. In some additional aspects, the bit map may consist of a plurality of dots organized in a pattern. In some examples, the plurality of dots may be organized in a series of hatch patterns, which may be angled from about 0° to about 45° and may be offset or shifted.

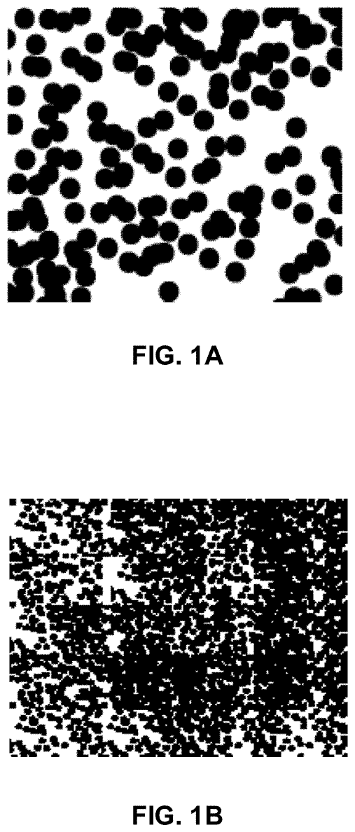

FIGS. 1A-1B show an example of a bit map that consists of a plurality of dots organized randomly on the bit map. FIG. 1A shows a zoomed-in view of the plurality of dots on the bitmap. FIG. 1B shows a zoomed-out view of the plurality of dots on the bitmap.

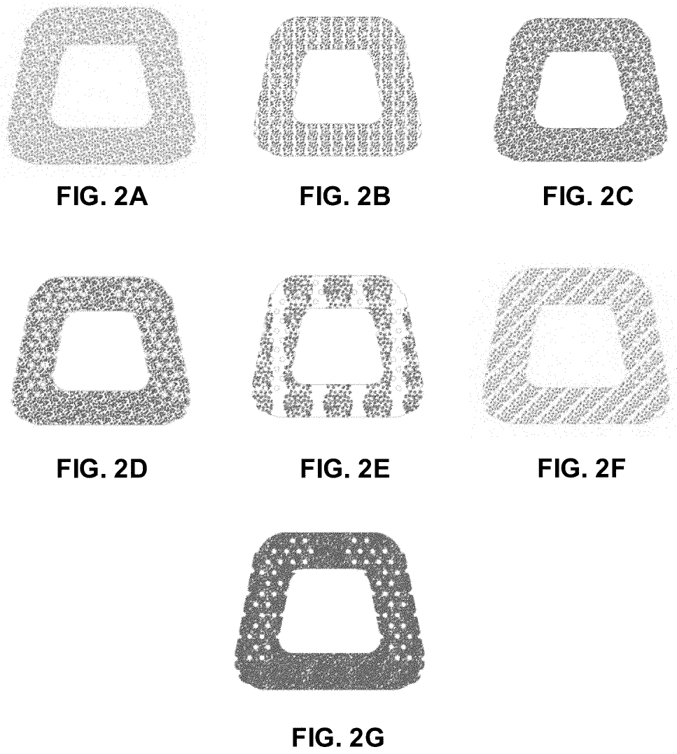

FIGS. 2A-2G show an example of a bit map that consists of a plurality of dots organized into various different patterns. FIG. 2A shows a 0° hatch pattern. FIG. 2B shows a 0° shifted hatch pattern. FIG. 2C shows a 12° hatch pattern. FIG. 2D shows a 19° hatch pattern. FIG. 2E shows a 0° shifted hatch. FIG. 2F shows a 45° hatch. FIG. 2G shows a fully-textured hatch.

FIGS. 10A-11B show the surface roughness of an implant made by laser etching the implant while it is in the green state. As can be seen in FIGS. 10A and 11A, the surface of the implant varies in height across a wide area of the implant. FIGS. 10B and 11B show the height profile along a linear path through the area shown in FIGS. 10A and 11B, respectively.

### Porosity

The method disclosed herein includes increasing the porosity of a silicon nitride green body. Increasing the porosity while the silicon nitride is a green body is beneficial for at least two reasons. First, it preserves the micro- and nano-structured surface topography that is generated during sintering and hot isostatic pressing. Second, it is more cost-effective because the green body is softer than a densified ceramic, making it easier to machine and etch. In some examples, machining and etching in the green state may cost 90% less compared to a densified ceramic. The pores in the surface of the completed implant may serve as, for example, sites for integration of osseous tissue or reservoirs or pockets for an osteogenic coating. In some embodiments, the pores may be orthogonal to one another, side-by-side, or randomly interspaced. In some aspects, the pores may align with other structural features of the implant, including surface features or teeth. In some aspects, the pores may be formed at an angle in the implant. In some additional embodiments, the pores may be uniform in size or may have different sizes. In yet additional embodiments, the pores may be aligned to go through the geometric center of the implant. In some embodiments, the pores may be formed by 3D-micro or laser-machining. In some aspects, the pores may be formed by peck drilling or laser etching.

In some embodiments, the pores may each have a diameter of about 300 μm to about 600 μm. In some aspects, the pores may each have a diameter of about 300 μm to about 325 μm, about 325 μm to about 350 μm, about 350 μm to about 375 μm, about 375 μm to about 400 μm, about 400 μm to about 425 μm, about 425 μm to about 450 μm, about 450 μm to about 475 μm, about 475 μm to about 500 μm, about 500 μm to about 525 μm, about 525 μm to about 550 μm, about 550 μm to about 575 μm, or about 575 μm to about 600 μm. In some additional aspects, the pores may each have a diameter of about 325 μm to about 550 μm, about 350 μm to about 500 μm, or about 375 μm to about 450 μm. In yet additional aspects, the pores may each have a diameter of about 300 μm, 325 μm, 350 μm, 375 μm, 400 μm, 425 μm, 450 μm, 475 μm, 500 μm, 525 μm, 550 μm, 575 μm, or about 600 μm. In some examples, the pores have a diameter of about 400 μm.

In some embodiments, the pores may each have a depth of at least 100 μm. In some embodiments, the pores are made by peck drilling to a depth of about 0.050 mm to about 0.500 mm at a time. In some aspects, the pores are made by peck drilling to a depth of about 0.050 mm, 0.060 mm, 0.070 mm, 0.080 mm, 0.090 mm, 0.100 mm, 0.150 mm, 0.200 mm, 0.250 mm, 0.300 mm, 0.350 mm, 0.400 mm, 0.450 mm, or about 0.500 mm at a time. In some examples, the pore can form an aperture in the implant.

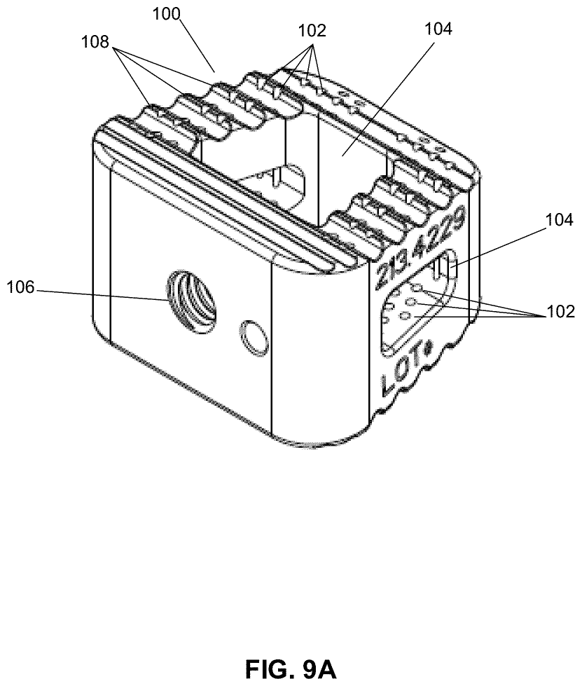

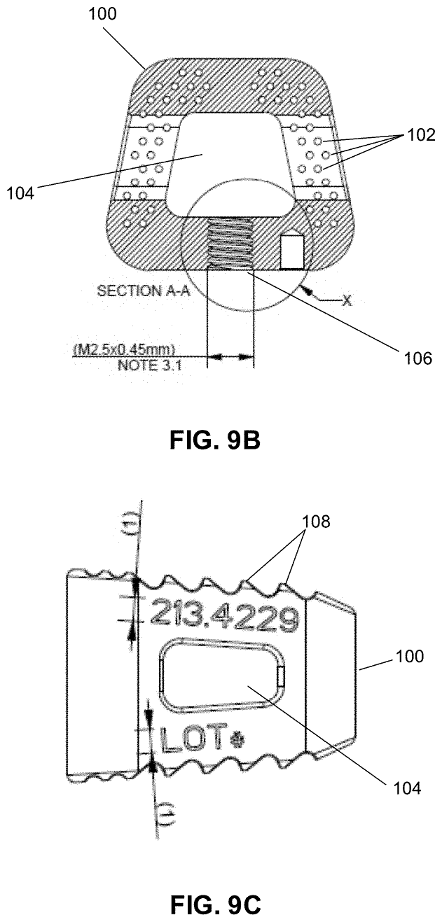

FIGS. 9A-9C show an example of an implant 100 with pores 102 formed by peck drilling.

### Coating

In some embodiments, the method may further comprise coating the implant after densification. Without being bound by theory, the coating may enhance osteoblastic activity by release of ions into the local environment, leading to accelerated fusion and enhanced fixation of the implant. In some embodiments, the coating may be a slurry and the coating may be applied to the implant through dip coating, spray coating, painting, physical vapor deposition, or other coating methods known in the art. The coating may later be fired after being applied to the implant. In some aspects, the coating may include SiYAION, NanoHA®, 45S5 Bioglass®, hydroxyapatite, and combinations thereof. In yet additional aspects, the coating may be uniform over the surface of the implant.

In some embodiments, the coating may have a thickness of between about 1 μm to about 50 μm. In some aspects, the coating may have a thickness of between about 1 μm to about 5 μm, 5 μm to about 10 μm, 10 μm to about 15 μm, 15 μm to about 20 μm, 20 μm to about 25 μm, 25 μm to about 30 μm, 30 μm to about 35 μm, 35 μm to about 40 μm, 40 μm to about 45 μm, or 45 μm to about 50 μm. In some additional aspects, the coating may have a thickness of about 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, or about 50 μm.

### Implant

Further described herein is a silicon nitride implant made by the methods described above. In some embodiments, the implant may be formed from a silicon nitride-doped ceramic. The implant may include biomedical implants, such as intervertebral spacers or other spinal implants, craniomaxillofacial implants, orthopedic screws, plates, or other fixation devices, articulation implants in the spine, hip, knee, shoulder, ankle or phalanges, implants for facial or other reconstructive plastic surgery, dental implants, and the like.

In preferred embodiments, the implant may be treated so as to improve its osteoconductive characteristics, antibacterial characteristics, and/or other desirable characteristics. This may be done by increasing the surface roughness of the implant as described herein, increasing the porosity of the implant as described herein, coating the implant, adding a filler or matrix to the implant, or other methods known in the art.

An example of an implant made by the methods described herein is shown in FIGS. 9A-9C. Although not visible in the figures, the surface of each of the implants depicted in FIGS. 9A-9C has been roughened by the methods described herein.

FIG. 9A depicts perspective view of a spinal implant 100. The implant 100 has a top with surface features or teeth 108 that improve osseointegration. The implant 100 includes openings 104 and a thread 106. The implant 100 also includes pores 102 formed by peck-drilling and/or lasers. Some of the pores 102 form apertures in the implant 100, while others terminate at a predetermined depth. In the depicted embodiment, the pores are aligned with the ridges 108.

FIG. 9B depicts a top-down view of a spinal implant 100. The implant 100 includes a roughened surface (hatched area) and a flat surface (white area). The implant 100 also includes an opening 104 and a thread 106. The implant also includes pores 102 formed by peck-drilling and/or lasers. In the depicted embodiment, the pores 102 are arranged on the left and right side of the implant 100 in a pattern.

FIG. 9C depicts a side view of a spinal implant 100. The implant 100 has a top with surface features or teeth 108 and an opening 104.

### EXAMPLES

### Example 1

Si₃N₄ has the ability to enhance osteogenesis and osteoconductivity due to its elutable surface chemistry. In simple terms, Si₃N₄ is thermodynamically unstable at homeostatic conditions. It is prone to react with water to form silicic acid (Si(OH)₄) and ammonia (NH₃) in accordance with the following chemical reaction:

Si₃N₄+12H₂O→3Si(OH)₄+4NH₃ ΔG=−565 kJ/mol (1)

The presence of bioavailable silicon in the form of silicic acid enhances osteogenic activity and various nitrogen-based moieties can either be mild disinfectants or powerful oxidants that disrupt prokaryotic cell function. However, other factors also likely aid in improving the material's osteoconductivity. These factors include surface charge, wettability, and phase chemistry. Si₃N₄ has a large negative surface charge (−45 mV to −70 mV) compared to PEEK (≈−50 mV) and Ti (−15 mV). Biomaterial surfaces possessing significant negative charge have been associated with higher serum protein adsorption and the upregulation of osteoblastic activity. The hydrophilicity of Si₃N₄ has been shown to be superior to PEEK and Ti with water contact angles of 8° to 66° (depending on surface treatment), 86°, and 71°, respectively. Hydrophilicity is positively correlated with negative surface charge and research has confirmed that readily wetted biomaterials lead to earlier and more effective bone apposition than hydrophobic compounds. It was also found that the phase chemistry of Si₃N₄ played a role in its osteoconductivity with osteoblasts preferably adhering and proliferating on various apatite, silicon-oxynitride, and SiYAION phases. Heat-treatments such as non-adiabatic cooling after hot-isostatic pressing, annealing in nitrogen (i.e., N₂-annealing), or thermal oxidation were effective in bringing these phases to the surface of the ceramic. A post-densification coating (i.e., glaze) using a SiYAION composition also led to enhanced osteoblastic activity.

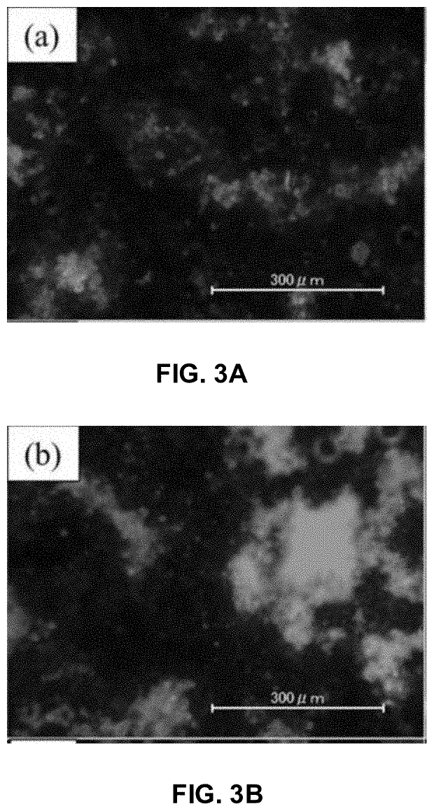

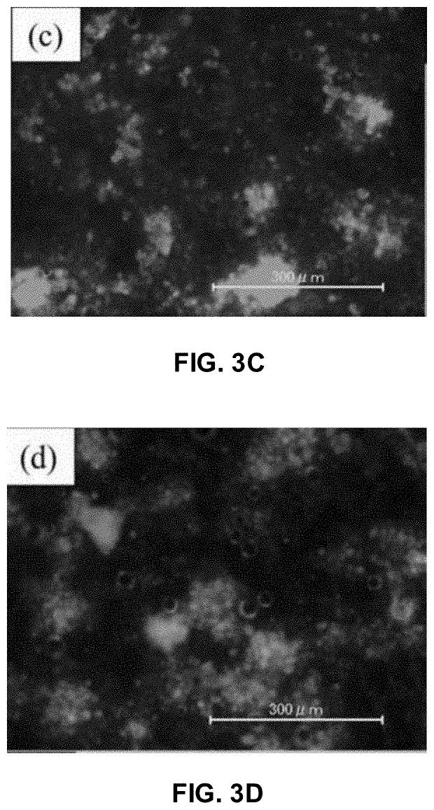

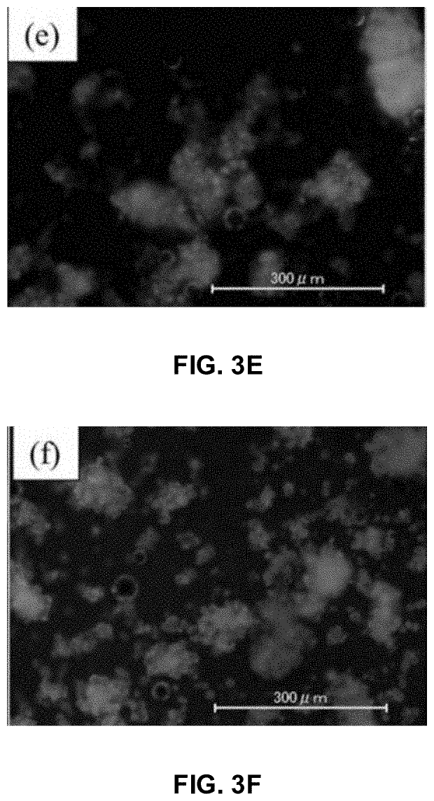

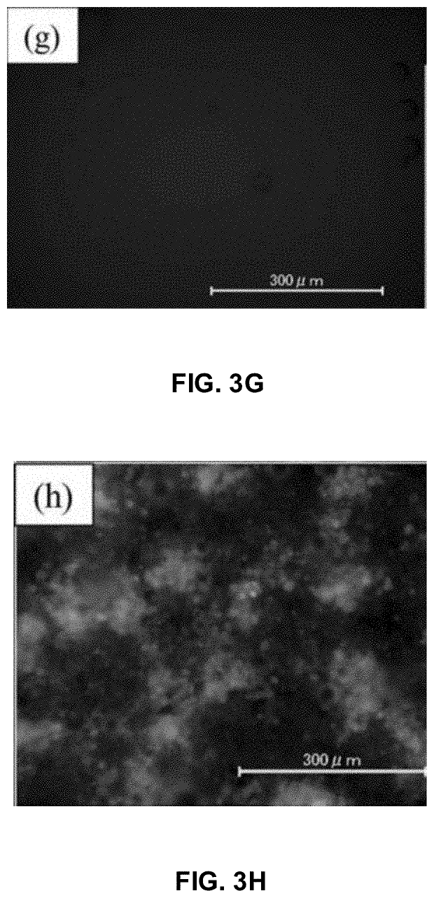

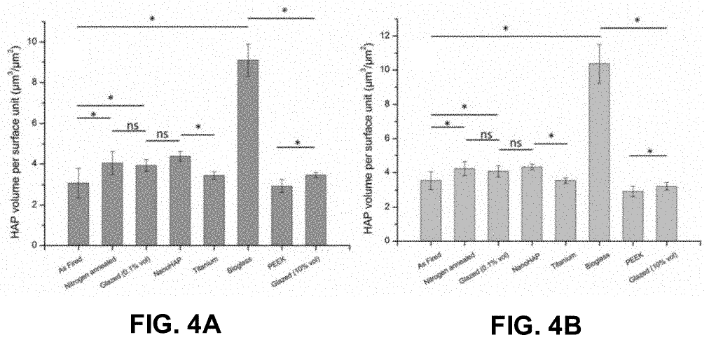

A comparative in vitro experiment was conducted in order to assess which of the various Si₃N₄ treatments was most effective in promoting osteoconductivity. The experiment involved culturing and incubating SaOS-2 osteosarcoma cells within an osteogenic medium for 7-days (with a media change every three days) on the following surfaces: (i) As-fired Si₃N₄; (ii) N₂-annealed Si₃N₄; (iii) 0.1 vol. % SiYAION glazed Si₃N₄; (iv) NanoHA® coated Si₃N₄; (v) machined titanium; (vi) 45S5 Bioglass®, (vii) PEEK; and 10 vol. %. SiYAION glazed Si₃N₄. After incubation, fluorescence microscopy was employed for measurement of cell proliferation and osteocalcin production. The amount of HAp formation by osteoblastic action was recorded via laser microscopy by two independent operators. The results of this unpublished work are shown in FIGS. 3A-3H and 4.

As indicated in FIGS. 3A-3H, all of the samples showed the presence of osteocalcin (i.e., a marker for osteoblastic activity) except for PEEK. Qualitatively, the largest and most uniform amount of osteocalcin production appeared to be on the 10 vol. % SiYAION, then the 45S5 Bioglass®, followed by the 0.1 vol. % SiYAION, and NanoHA® surfaces. Poorer distribution and/or lower deposition volumes were noted on the N₂-annealed, as-fired, and Ti samples in that order.

Results for HAp deposition reasonably confirmed the osteocalcin data (see FIGS. 4A-4B) except for the 45S5 Bioglass®. Although it had a surprisingly large amount of HAp, this result was obtained using a solid disc and therefore may not be representative of an actual coating. The NanoHA® showed the next average highest deposition volume, followed by N₂-annealed Si₃N₄, and the two SiYAION glazed samples. There were no statistical differences between these samples. The as-fired Si₃N₄ and Ti samples were statistically equivalent in HAp volume, and both were superior to PEEK. Collectively, these results suggest that a coating such as 45S5 Bioglass® or NanoHA® may be reasonable choices for improving the osteoconductivity of as-fired Si₃N₄. They can be applied at low or ambient temperatures whereas the SiYAION glazes require 1400° C. Unfortunately, neither N₂-annealing nor SiYAION glazing may be preferred because thermal cycling to this temperature results in de-sintering (or bloating) of Si₃N₄. In turn, this leads to a reduction of both bulk and as-fired flexural strengths (i.e., between ˜13% and ˜18%). However, it may be possible to apply the SiYAION glaze using laser sintering/melting. Localized surface heating should not negatively affect bulk material properties.

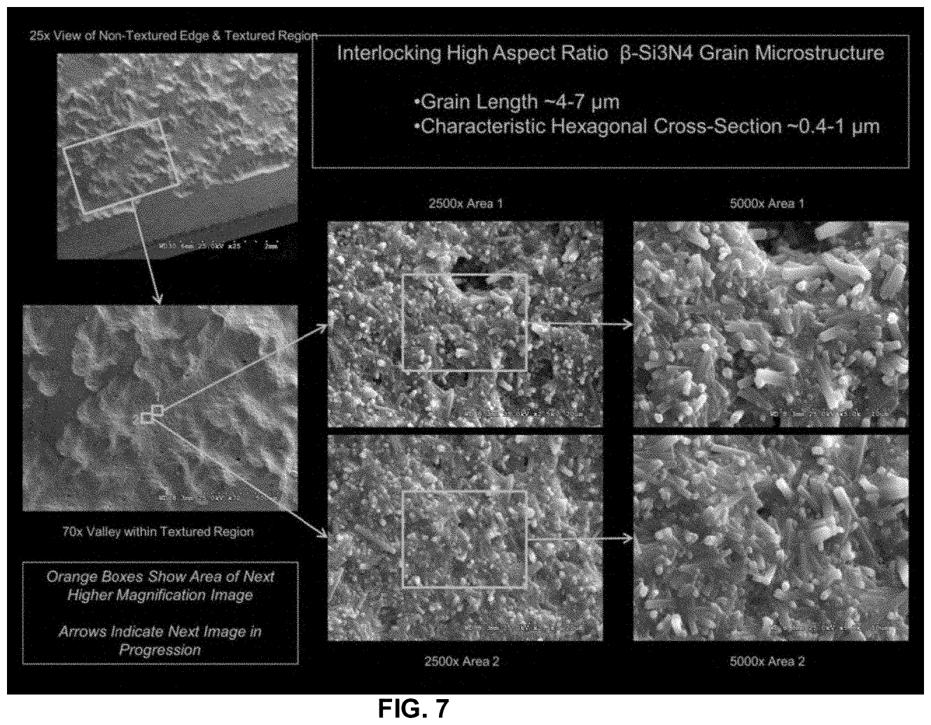

While previously Ti-alloys and PEEK have substantiated the importance of topography in appositional healing, this phenomenology was only recently demonstrated for Si₃N₄. However, Si₃N₄'s current topographical features are only apparent at the micron and sub-micron scales. As shown in FIG. 7, Si₃N₄'s as-fired surface structure consists of anisotropic grains that are typically 1 μm× up to 10 μm with individual features (i.e., asperities, sharp corners, points, pits, pockets, and grain intersections) that can range in size from <100 nm to 1 μm. While this structure is morphologically different from surface-functionalized titanium, it has some common features (e.g., sharp corners, points, and pockets). Detailed mechanistic studies have yet to be conducted, but it is believed that these types of features in Si₃N₄ may contribute to appositional bone healing in a similar way as in functionalized titanium.

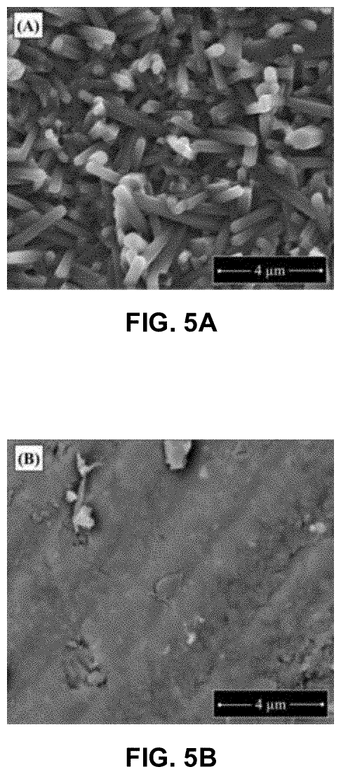

While the prior research for Ti-alloys and PEEK has substantiated the importance of topography in appositional healing, this phenomenology was only recently demonstrated for Si₃N₄. However, Si₃N₄'s current topographical features are only apparent at the micron and sub-micron scales. As shown in FIG. 5A, Si₃N₄'s as-fired surface structure consists of anisotropic grains that are typically 1 μm× up to 10 μm with individual features (i.e., asperities, sharp corners, points, pits, pockets, and grain intersections) that can range in size from <100 nm to 1 μm. While this structure is morphologically different from surface-functionalized titanium, it has some common features (e.g., sharp corners, points, and pockets).

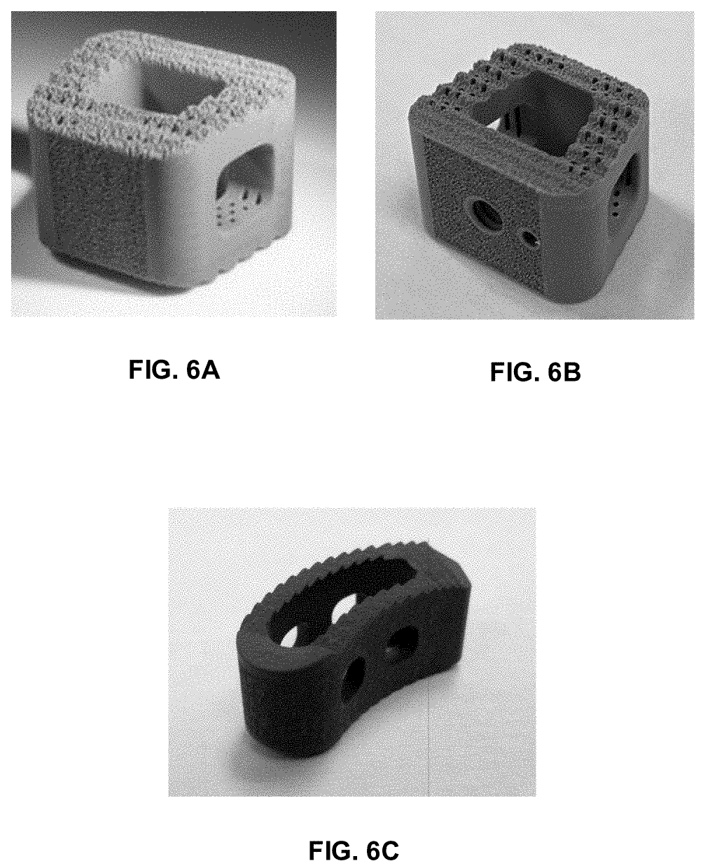

Nevertheless, current Si₃N₄ intervertebral spinal spacers do not have the broad range of surface topography that has been engineered into state-of-the-art titanium spacers. In contrast to the optimum surface roughness found for Ti-alloy implants of Rₐ=3 to 4 μM, Si₃N₄'S as-fired surface finish was found to only be in the range of 0.34 μm 1.0 μm. However, laser texturing has been employed as a method of increasing the macro-surface roughness of Si₃N₄ implants. Examples of a textured implant are shown in FIGS. 6A-6C.

FIG. 7 provides a collage of scanning electron micrographs at increasingly higher magnifications which highlight the topographical features of this prototype. The average surface roughness of this implant was dramatically increased to ˜43.5 μm. This change in roughness may be excessive, but the result suggests that the process has the potential to achieve a targeted value of Rₐ<10 μm and preferred range of between 3 and 4 μm.

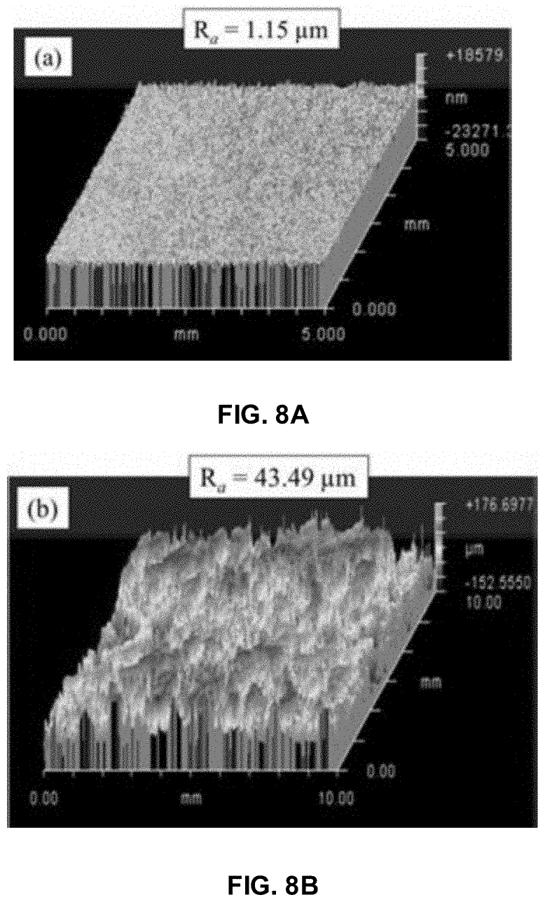

One method of increasing roughness is by laser etching of implants in their “green state” (i.e., prior to densification). Doing so will preserve their micro- and nano-structure which is formed during firing. For instance, shown in FIGS. 8A and 8B are white-light interferometry measurements of an as-fired Si₃N₄ surface and HIPed and laser-etched Si₃N₄ surface.

Two points are pertinent in these graphs: (i) The as-fired surface consists only of acicular protruding Si₃N₄ grains. There are no intermediate or macro-rough features. Note that the average roughness is Rₐ=1.15 μm; and (ii) The laser-etched surface adds micro- and macro-rough texture. The average roughness of this surface was Rₐ=43.49 μm (i.e., 38× coarser than the as-fired surface). While this increase may be too large for appositional healing, the results certainly demonstrate that a broad roughness range is possible.

What is claimed is:

1. A method for manufacturing a silicon nitride implant, the method comprising:
providing a silicon nitride green body;
increasing the surface roughness of the silicon nitride green body;
increasing the porosity of the silicon nitride green body; and
sintering the silicon nitride green body to obtain the silicon nitride implant.

2. The method of claim 1, wherein the step of increasing the surface roughness of the silicon nitride green body is performed by laser etching.

3. The method of claim 1, wherein the step of increasing the porosity of the silicon nitride green body is performed by peck drilling and/or laser etching.

4. The method of claim 1, wherein the silicon nitride implant has a Sₐ value of less than about 100 μm.

5. The method of claim 4, wherein the silicon nitride implant has a Sₐ value of about 50 μm to about 100 μm.

6. The method of claim 4, wherein the silicon nitride implant has a Sₐ value of about 60 μm to about 90 μm.

7. The method of claim 1, further comprising applying an osteogenic coating to the silicon nitride implant after the sintering step.

8. The method of claim 7, wherein the osteogenic coating is selected from the group consisting of SiYAION, NanoHA®, 45S5 Bioglass®, hydroxyapatite, and combinations thereof.

9. An implant formed by the method of claim 1.

10. A method for manufacturing a silicon nitride implant, the method comprising:
providing a silicon nitride green body;
laser etching an outer surface of the silicon nitride green body to increase the surface roughness of the silicon nitride green body;
peck drilling and/or laser etching the silicon nitride green body to create porosity in the silicon nitride green body; and
sintering the silicon nitride green body to obtain the silicon nitride implant.

11. The method of claim 10, wherein the silicon nitride implant has a Sₐ value of less than about 100 μm.

12. The method of claim 11, wherein the silicon nitride implant has a Sₐ value of about 50 μm to about 100 μm.

13. The method of claim 11, wherein the silicon nitride implant has a Sₐ value of about 60 μm to about 90 μm.

14. The method of claim 10, further comprising applying an osteogenic coating to the silicon nitride implant after the sintering step.

15. The method of claim 14, wherein the osteogenic coating is selected from the group consisting of SiYAION, NanoHA®, 45S5 Bioglass®, hydroxyapatite, and combinations thereof.

16. An implant formed by the method of claim 10.
